(19) United States
(12) Patent Application Publication
(10) Pub. No.: US 2017/0153761 A1
(43) Pub. Date: Jun. 1, 2017

(54) Touch Sensing Device and Mobile Apparatus

(71) Applicant: Boe Technology Group Co., Ltd., Beijing (CN)

(72) Inventor: Hanjun PARK, Beijing (CN)

(21) Appl. No.: 15/122,616

(22) PCT Filed: Sep. 23, 2015
(86) PCT No.: PCT/CN2015/090364

§ 371 Date: Aug. 30, 2016

(30) Foreign Application Priority Data
May 15, 2015 (CN) 201510247886.4

(51) Int. Cl. G06F 3/041; A61B 5/00; A61B 5/026; G06F 3/042; A61B 5/0205

(52) U.S. Cl. CPC G06F 3/0418; G06F 3/0412; G06F 3/042; A61B 5/02055; A61B 5/0261; A61B 5/6898; G06F 2203/04102; A61B 5/015

(57) ABSTRACT

Embodiments of the disclosure provide a touch sensing device, comprising a substrate and a sensor array arranged on the substrate, the sensor array comprising a plurality of sensor units. Each sensor unit comprises an infrared light emitting diode and an infrared sensing thin film transistor. The infrared light emitting diode is configured to emit infrared rays, and the infrared sensing thin film transistor is configured to detect infrared rays emitted from the infrared light emitting diode and reflected by a user, so as to generate a current signal capable of representing user body data. Embodiments of the disclosure further provide a mobile apparatus comprising the touch sensing device and a display device. The touch sensing device is arranged at a back side of the mobile apparatus, and the display device is arranged at a front side of the mobile apparatus and is configured to display the user body data.

### RELATED APPLICATIONS

The present application is the U.S. national phase entry of PCT/CN2015/090364, with an international filing date of Sep. 23, 2015, which claims the benefit of Chinese Patent Application No. 201510247886.4, filed May 15, 2015, the entire disclosures of which are incorporated herein by reference.

### TECHNICAL FIELD

The disclosure relates to sensing technology, particularly to a touch sensing device and a mobile apparatus.

### BACKGROUND

With the development of the sensing technology, various sensors with the sensing function that can be applied in medical and health care devices and wearable devices have come up. In addition, sensors that can be used in cooperation with a mobile apparatus have also come up. Such products generally have three parts, i.e., a sensor part, a display part and a communication part using BlueTooth/NFC/Wifi to transmit data to the mobile apparatus.

Although the development of the sensing technology brings convenience for human life, existing sensor devices generally adopt a single position and single time measuring manner, which results in a relatively low data accuracy.

In addition, for sensors used in cooperation with the mobile apparatus, because an additional communication part is required, the cost is increased, which results in a relatively high price for the user. Moreover, it also brings separate charging problem.

### SUMMARY

Therefore, it is desired to provide an improved touch sensing device and a mobile apparatus, which can mitigate or avoid one or more of the above problems.

According to one aspect of the disclosure, a touch sensing device is provided, comprising a substrate and a sensor array arranged on the substrate. The sensor array comprises a plurality of sensor units. Each sensor unit comprises an infrared light emitting diode and an infrared sensing thin film transistor. The infrared light emitting diode is configured to emit infrared rays. The infrared sensing thin film transistor is configured to detect infrared rays emitted from the infrared light emitting diode and reflected by a user, so as to generate a current signal capable of representing user body data.

As mentioned above, the existing sensor devices generally adopt a single position and single time measuring manner, which results in a relatively low data accuracy. By enabling the touch sensing device to comprise a sensor array and enabling each sensor unit in the sensor array to comprise an infrared light emitting diode and an infrared sensing thin film transistor, the measuring position and the measuring time can be selected based on specific applications and requirements, thereby realizing a multi-position and multi-time measuring manner, which improves the data accuracy significantly.

According to an embodiment of the disclosure, the user body data may comprise body temperature, pulse, blood flow rate, blood pressure and blood oxygen ratio.

For example, in the case of measuring body temperature of a user, by enabling the sensor unit to comprise an infrared light emitting diode and an infrared sensing thin film transistor for measuring the body temperature, a current signal capable of representing the body temperature of the user can be generated so as to obtain the body temperature of the user. In the case of measuring other body data of the user, by enabling the sensor unit to comprise an infrared light emitting diode and an infrared sensing thin film transistor for measuring other body data, a current signal capable of representing other body data of the user can be generated so as to obtain corresponding body data.

According to another embodiment of the disclosure, the plurality of sensor units may comprise various types of infrared sensing thin film transistors to generate various types of current signals, so as to represent various types of user body data.

By means of such a configuration, various types of body data can be obtained simultaneously so as to meet the user requirements better. For example, the sensor units located in different areas of the sensor array can comprise an infrared sensing thin film transistor for measuring body temperature, an infrared sensing thin film transistor for measuring pulse and an infrared sensing thin film transistor for measuring blood pressure respectively. In this way, various types of current signals representing body temperature, pulse and blood pressure can be generated simultaneously so as to obtain the body temperature, pulse and blood pressure data of the user simultaneously.

According to another embodiment of the disclosure, the number of the infrared sensing thin film transistors in each sensor unit is plural. The plurality of infrared sensing thin film transistors comprise a plurality of infrared sensing thin film transistor groups connected in parallel. Each infrared sensing thin film transistor group comprising a plurality of infrared sensing thin film transistors connected in series.

By connecting the plurality of infrared sensing thin film transistors in series so as to form an infrared sensing thin film transistor group and then connecting a plurality of such infrared sensing thin film transistor groups in parallel, a fine current can be obtained so as to provide higher detection accuracy.

According to another embodiment of the disclosure, each sensor unit may further comprise a switch element for controlling the infrared light emitting diode.

By arranging a switch element for controlling the infrared light emitting diode, turn-on and turn-off of the infrared light emitting diode can be controlled flexibly based on specific applications and requirements.

According to a further embodiment of the disclosure, the touch sensing device may further comprise a protective layer arranged on the sensor array.

By arranging a protective layer, the sensitive sensor area can be protected so as to reduce the failure rate thereof.

According to a further embodiment of the disclosure, the substrate comprised by the touch sensing device can be a glass substrate or a flexible substrate.

The type of the substrate can be selected based on specific applications and requirements. For example, a flexible sensor array can be realized on a flexible substrate so as to meet requirement in some applications.

According to a further embodiment of the disclosure, the plurality of sensor units of the sensor array can be arranged in a rectangular, circular or radial form.

Different arrangement manners of the sensor array are applicable for different applications and requirements.

According to another aspect of the disclosure, a mobile apparatus is provided, comprising the above touch sensing device and a display device. The touch sensing device is arranged at a back side of the mobile apparatus. The display device is arranged at a front side of the mobile apparatus and is configured to display user body data.

By integrating the touch sensing device comprising a sensor array to the back side of the mobile apparatus, the additional communication part can be eliminated, so as to reduce the cost. Moreover, a simple and convenient design can be provided for the user, and the separate charging problem is solved.

According to an embodiment of the disclosure, the mobile apparatus may further comprise a driving device. The driving device comprises: a first source signal driver, a first gate signal driver and an infrared light emitting diode driver for driving the touch sensing device; a second source signal driver and a second gate signal driver for driving the display device; and a timing controller. The timing controller is configured to control timing of the first and second source signal drivers, the first and second gate signal drivers and the infrared light emitting diode driver.

According to another embodiment of the disclosure, the mobile apparatus may further comprise a read out device. The read out device is configured to read out a current signal generated by the touch sensing device representing user body data.

### BRIEF DESCRIPTION OF THE DRAWINGS

Now, the concept and other advantages of the disclosure will be described with reference to the drawings through nonrestrictive embodiments.

FIG. 1 shows a circuit schematic view of a touch sensing device according to an embodiment of the disclosure.

FIG. 2 shows a circuit schematic view of a sensor unit in the touch sensing device according to an embodiment of the disclosure.

FIG. 3 shows a longitudinal sectional view of a sensor unit in the touch sensing device according to an embodiment of the disclosure.

FIGS. 4A and 4B show schematic views of a mobile apparatus according to an embodiment of the disclosure.

FIG. 5 shows a driving schematic view of a touch sensing device and a display device in the mobile apparatus according to an embodiment of the disclosure.

### DETAILED DESCRIPTION

Next, the specific examples of a display substrate, a display device and a manufacturing method of the display substrate according to embodiments of the disclosure will be explained exemplarily with reference to the drawings. The drawings are schematic and are not drawn proportionally. Moreover, they are only for explaining the embodiments of the disclosure rather than intending to limit the protection scope of the disclosure.

It should be understood that when it is stated that an element or a layer is “on” another element or layer, “connected to” or “coupled to” to another element or layer, it can be directly on, connected to or coupled to another element or layer, or an inserted element or layer may also exist. On the contrary, when it is stated that an element “is directly on”, “directly connected to” or “directly coupled to” another element or layer, no inserted element or layer exists.

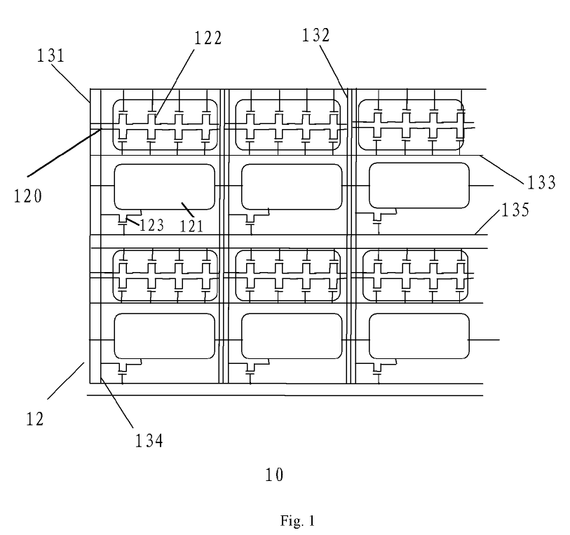

FIG. 1 shows a circuit schematic view of a touch sensing device according to an embodiment of the disclosure. As shown in FIG. 1, a touch sensing device 10 according to an embodiment of the disclosure comprises a substrate (not shown) and a sensor array 12 arranged on the substrate. The sensor array 12 comprises a plurality of sensor units 120. Each sensor unit 120 comprises an infrared light emitting diode 121 and an infrared sensing thin film transistor 122. The infrared light emitting diode 121 is configured to emit infrared rays. The infrared sensing thin film transistor 122 is configured to detect infrared rays emitted from the infrared light emitting diode 121 and reflected by a user, so as to generate a current signal capable of representing user body data.

As mentioned above, the existing sensor devices generally adopt a single position and single time measuring manner, which results in a relatively low data accuracy. By enabling the touch sensing device to comprise a sensor array and enabling each sensor unit in the sensor array to comprise an infrared light emitting diode and an infrared sensing thin film transistor, the measuring position and the measuring time can be selected based on specific applications and requirements. For example, a plurality of sensor units at a plurality of predetermined positions in the sensor array can perform sensing at a plurality of predetermined time points based on needs. Compared with the conventional single position and single time measuring manner, it can realize a multi-position and multi-time measuring manner, thereby improving the data accuracy significantly.

The user body data may comprise body temperature, pulse, blood flow rate, blood pressure and blood oxygen ratio. For example, in the case of measuring body temperature of a user, by enabling the sensor unit to comprise an infrared light emitting diode and an infrared sensing thin film transistor for measuring the body temperature, a current signal capable of representing the body temperature of the user can be generated so as to obtain the body temperature data of the user. In the case of measuring other body data of the user, by enabling the sensor unit to comprise an infrared light emitting diode and an infrared sensing thin film transistor for measuring other body data, a current signal capable of representing other body data of the user can be generated so as to obtain corresponding body data.

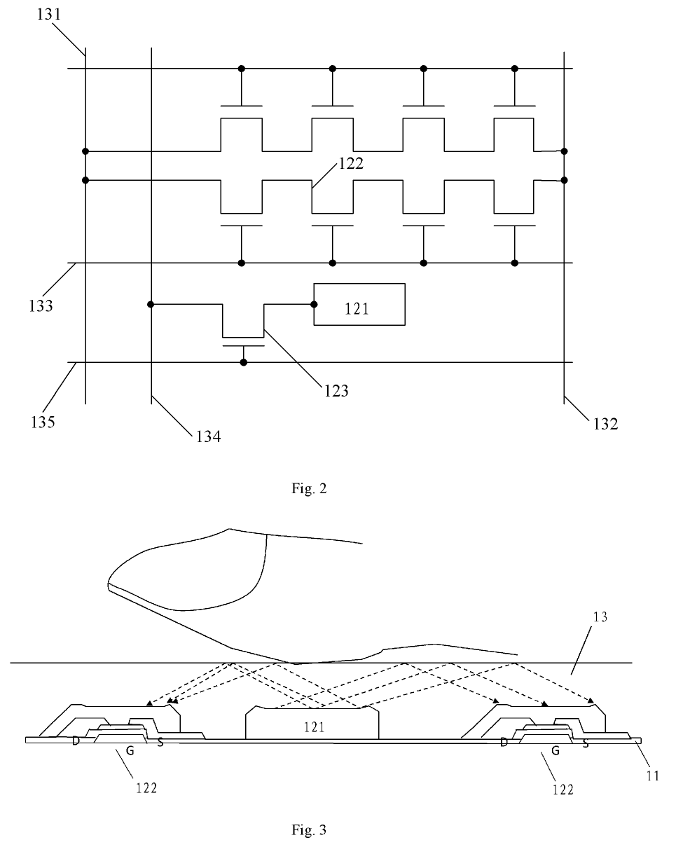

FIG. 2 shows a circuit schematic view of a sensor unit in the touch sensing device according to an embodiment of the disclosure. As shown in FIG. 1 and FIG. 2, further, each sensor unit 120 in the sensor array 12 may further comprise a switch element 123 for controlling the infrared light emitting diode 121. By arranging a switch element for controlling the infrared light emitting diode, turn-on and turn-off of the infrared light emitting diode can be controlled flexibly based on specific applications and requirements.

In FIG. 1 and FIG. 2, the switch element 123 is for example a switch transistor. However, the disclosure is not limited to this. The skilled person in the art can select the switch element based on specific applications and requirements, as long as it can control turn-on and turn-off of the infrared light emitting diode.

As shown in FIG. 1 and FIG. 2, further, the touch sensing device 10 may further comprise a thin film transistor source line 131, a thin film transistor read out line 132, a thin film transistor gate line 133, a light emitting diode switch element source line 134 and a light emitting diode switch element gate line 135 arranged in the sensor array. By means of the respective signal lines arranged in the sensor array, the desired addressing manner of the sensor unit can be achieved. For example, a plurality of sensor units at a plurality of predetermined positions in the sensor array can be addressed at a plurality of predetermined time points, so as to enable the infrared light emitting diodes in the plurality of sensor units to turn on and enable the corresponding infrared sensing thin film transistors to perform sensing, thereby realizing the multi-position and multi-time measuring manner.

According to an embodiment of the disclosure, the number of the infrared sensing thin film transistors in each sensor unit can be plural. The plurality of infrared sensing thin film transistors comprise a plurality of infrared sensing thin film transistor groups connected in parallel. Each infrared sensing thin film transistor group comprising a plurality of infrared sensing thin film transistors connected in series.

For example, as shown in FIG. 1 and FIG. 2, the number of the infrared sensing thin film transistors 122 in the sensor unit 120 is eight. The eight infrared sensing thin film transistors comprise two infrared sensing thin film transistor groups connected in parallel. Each infrared sensing thin film transistor group comprises four infrared sensing thin film transistors connected in series.

By connecting the plurality of infrared sensing thin film transistors in series so as to form an infrared sensing thin film transistor group and then connecting a plurality of such infrared sensing thin film transistor groups in parallel, a fine current can be obtained so as to provide higher detection accuracy.

It should be noted that FIG. 1 and FIG. 2 are only exemplary embodiments of the disclosure rather than limitations to the disclosure. The number of the infrared sensing thin film transistors in each sensor unit can be one, and can also be plural. The skilled person in the art can select the specific number and connecting manner of the infrared sensing thin film transistors in each sensor unit based on applications and requirements.

According to another embodiment of the disclosure, the plurality of sensor units may comprise various types of infrared sensing thin film transistors to generate various types of current signals, so as to represent various types of user body data. By means of such a configuration, various types of body data can be obtained simultaneously so as to meet the user requirements better.

For example, the sensor units located in different areas of the sensor array can comprise an infrared sensing thin film transistor for measuring body temperature, an infrared sensing thin film transistor for measuring pulse and an infrared sensing thin film transistor for measuring blood pressure respectively. In this way, various types of current signals representing body temperature, pulse and blood pressure can be generated simultaneously so as to obtain the body temperature, pulse and blood pressure data of the user simultaneously.

According to a further embodiment of the disclosure, the plurality of sensor units of the sensor array can be arranged in a rectangular, circular or radial form. Different arrangement manners of the sensor array are applicable for different applications and requirements.

FIG. 3 shows a longitudinal sectional view of a sensor unit in the touch sensing device according to an embodiment of the disclosure. As shown in FIG. 3, the sensor unit comprises an infrared light emitting diode 121 and an infrared sensing thin film transistor 122 arranged on a substrate 11. The infrared light emitting diode 121 is configured to emit infrared rays. The infrared sensing thin film transistor 122 is configured to detect infrared rays emitted from the infrared light emitting diode 121 and reflected by a user (here the infrared rays are reflected by user fingers), so as to generate a current signal capable of representing user body data. In FIG. 3, the symbols D, S, G represent a drain, a source and a gate of the infrared sensing thin film transistor 122 respectively.

Further, as shown in FIG. 3, the touch sensing device may further comprise a protective layer 13 arranged on the sensor array. The protective layer for example can be a transparent conductive protective layer. By arranging a protective layer, the sensitive sensor area can be protected, so as to reduce failure rate thereof.

Further, the substrate 11 comprised by the touch sensing device can be a glass substrate or a flexible substrate. The type of the substrate can be selected based on specific applications and requirements. For example, a flexible sensor array can be realized on a flexible substrate so as to meet requirements in some applications.

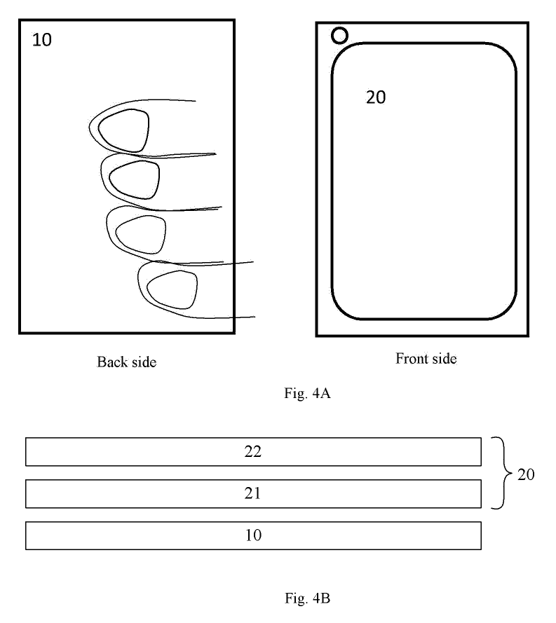

FIGS. 4A and 4B show schematic views of a mobile apparatus according to an embodiment of the disclosure. FIG. 4A shows a front side and a back side of the mobile apparatus. FIG. 4B shows a longitudinal configuration of the mobile apparatus.

As shown in FIG. 4A, the mobile apparatus comprises the touch sensing device 10 as stated above and a display device 20. The touch sensing device 10 is arranged at a back side of the mobile apparatus (the user fingers as shown in the figure indicate that the touch sensing device 10 is touchable by the user). The display device 20 is arranged at a front side of the mobile apparatus and is configured to display user body data.

The mobile apparatus for example can be a mobile phone, a laptop, a panel computer etc. The display device comprised by the mobile apparatus for example can be a liquid crystal display device, an OLED display device etc.

As shown in FIG. 4B, the display device 20 is arranged on the touch sensing device 10 located at the back side of the mobile apparatus. In the event that the display device 20 is a liquid crystal display device, an array substrate 21 and a color film substrate 22 of the display device 20 can be arranged on the touch sensing device 10 respectively. The required electrical connection between the touch sensing device 10 and the display device 20 can be realized through for example a flexible circuit board (FPC), an integrated circuit (IC) or a printed circuit board (PCB).

By integrating the touch sensing device comprising a sensor array to the back side of the mobile apparatus, the additional communication part can be eliminated, so as to reduce the cost. Moreover, a simple and convenient design can be provided for the user, and the separate charging problem is solved.

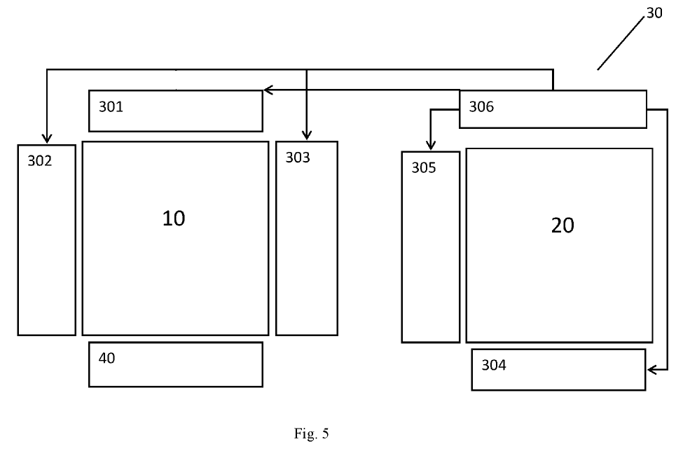

FIG. 5 shows a driving schematic view of a touch sensing device and a display device in the mobile apparatus according to an embodiment of the disclosure. As shown in FIG. 5, the mobile apparatus may further comprise a driving device 30. The driving device 30 comprises: a first source signal driver 301, a first gate signal driver 302 and an infrared light emitting diode driver 303 for driving the touch sensing device 10; a second source signal driver 304 and a second gate signal driver 305 for driving the display device 20; and a timing controller 306. The timing controller 306 is configured to control timing of the first and second source signal drivers, the first and second gate signal drivers and the infrared light emitting diode driver. The driving device 30 can be realized through for example a flexible circuit board (FPC), an integrated circuit (IC) or a printed circuit board (PCB) based on specific applications and requirements.

Further, as shown in FIG. 5, the mobile apparatus may further comprise a read out device 40. The read out device 40 is configured to read out a current signal generated by the touch sensing device 10 representing user body data. The read out device 40 can be realized through for example a flexible circuit board (FPC), an integrated circuit (IC) or a printed circuit board (PCB) based on specific applications and requirements.

Further, the mobile apparatus for example may further comprise a memory and a processor (not shown in the figure). The memory can store a plurality of user body data corresponding to different current signals generated by the touch sensing device, so that the corresponding user body data can be obtained by access to the memory. For example, the memory can store a user body data lookup table corresponding to different current signals generated by the touch sensing device. The corresponding user body data can be obtained by querying the lookup table. The processor is used for controlling operations of the touch sensing device, the display device, the driving device, the read out device and the memory.

Take the example that the user body data is the body temperature of the user. When the user touches the touch sensing device 10, the processor can control the driving device 30 and the touch sensing device 10, so as to enable the touch sensing device 10 to perform sensing thereby generating a current signal representing the body temperature of the user. Further, the processor can control the read out device 40 to enable it to read out the current signal generated by the touch sensing device 10. Then, the body temperature of the user can be obtained by access to the memory. Further, the processor can control the driving device 30 and the display device 20 so that the display device 20 can display the body temperature of the user.

Although the exemplary embodiments of the disclosure have been described in detail with reference to the drawings, such description should be regarded as illustrative or exemplary rather than restrictive. The disclosure is not limited to the disclosed embodiments. Different embodiments described above and in the claims can also be combined. The skilled person in the art, when carrying out the disclosure as claimed, can understand and carry out other modifications of the disclosed embodiments based on the study on the drawings, the description and the claims. These modifications also fall within the protection scope of the disclosure.

In the claims, the word “comprise” does not exclude presence of other components or steps. The fact that several technical measures have been recited in mutually different dependent claims does not mean that the combination of these technical measures cannot be utilized advantageously.

1. A touch sensing device, comprising a substrate and a sensor array arranged on the substrate, the sensor array comprising a plurality of sensor units,
wherein each sensor unit comprises an infrared light emitting diode and an infrared sensing thin film transistor, and wherein the infrared light emitting diode is configured to emit infrared rays, the infrared sensing thin film transistor is configured to detect infrared rays emitted from the infrared light emitting diode and reflected by a user, so as to generate a current signal capable of representing user body data.

2. The touch sensing device according to claim 1, wherein the user body data comprises body temperature, pulse, blood flow rate, blood pressure and blood oxygen ratio.

3. The touch sensing device according to claim 1, wherein the plurality of sensor units comprise various types of infrared sensing thin film transistors to generate various types of current signals, so as to represent various types of user body data.

4. The touch sensing device according to claim 1, wherein the number of the infrared sensing thin film transistors in each sensor unit is plural, and the plurality of infrared sensing thin film transistors comprise a plurality of infrared sensing thin film transistor groups connected in parallel, each infrared sensing thin film transistor group comprising a plurality of infrared sensing thin film transistors connected in series.

5. The touch sensing device according to claim 1, wherein each sensor unit further comprises a switch element for controlling the infrared light emitting diode.

6. The touch sensing device according to claim 1, further comprising a protective layer arranged on the sensor array.

7. The touch sensing device according to claim 1, wherein the substrate is a glass substrate or a flexible substrate.

8. The touch sensing device according to claim 1, wherein the plurality of sensor units of the sensor array are arranged in a rectangular, circular or radial form.

9. A mobile apparatus, comprising the touch sensing device according to claim 1 and a display device,
wherein the touch sensing device is arranged at a back side of the mobile apparatus, and the display device is arranged at a front side of the mobile apparatus and is configured to display user body data.

10. The mobile apparatus according to claim 9, wherein the mobile apparatus further comprises a driving device, the driving device comprising:
a first source signal driver, a first gate signal driver and an infrared light emitting diode driver for driving the touch sensing device;
a second source signal driver and a second gate signal driver for driving the display device; and
a timing controller,
wherein the timing controller is configured to control timing of the first and second source signal drivers, the first and second gate signal drivers and the infrared light emitting diode driver.

11. The mobile apparatus according to claim 9, wherein the mobile apparatus further comprises a read out device, the read out device being configured to read out a current signal generated by the touch sensing device representing user body data.

12. The mobile apparatus according to claim 9, wherein the user body data comprises body temperature, pulse, blood flow rate, blood pressure and blood oxygen ratio.

13. The mobile apparatus according to claim 9, wherein the plurality of sensor units comprise various types of infrared sensing thin film transistors to generate various types of current signals, so as to represent various types of user body data.

14. The mobile apparatus according to claim 9, wherein the number of the infrared sensing thin film transistors in each sensor unit is plural, and the plurality of infrared sensing thin film transistors comprise a plurality of infrared sensing thin film transistor groups connected in parallel, each infrared sensing thin film transistor group comprising a plurality of infrared sensing thin film transistors connected in series.

15. The mobile apparatus according to claim 9, wherein each sensor unit further comprises a switch element for controlling the infrared light emitting diode.

16. The mobile apparatus according to claim 9, further comprising a protective layer arranged on the sensor array.

17. The mobile apparatus according to claim 9, wherein the substrate is a glass substrate or a flexible substrate.

18. The mobile apparatus according to claim 9, wherein the plurality of sensor units of the sensor array are arranged in a rectangular, circular or radial form.
